(19) United States
(12) Patent Application Publication
(10) Pub. No.: US 2002/0010155 A1
(43) Pub. Date: Jan. 24, 2002

(54) Novel process

(75) Inventors: Mark Jason Hughes, Welwyn (GB); John Kitteringham, Hertford (GB); Victor Witold Jacewicz, Southborough (GB); Gillian Elizabeth Smith, Bishop's Stortford (GB); Martyn Voyle, Welwyn (GB); Evgeny Shapiro, Haifa Bay (IL)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 09/922,063

(22) Filed: Aug. 3, 2001

Related U.S. Application Data
(63) Continuation of application No. 09/564,485, filed on May 4, 2000, now abandoned.
Application No. 09/564,485 is a continuation of application No. 09/102,443, filed on Jun. 22, 1998, now abandoned.

(30) Foreign Application Priority Data
Jun. 20, 1997 (GB) 9713097.5

(51) Int. Cl. A61K 31/695; A61K 31/445; C07D 211/08

(52) U.S. Cl. 514/63; 514/317; 514/330; 546/14; 546/192; 546/226

(57) ABSTRACT

A process for the preparation of a 4-aryl-3-oxymethyl-piperidine of structure (1)

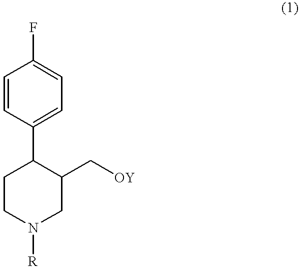

in which R is hydrogen or an alkyl, arylalkyl, allyl, acyl, carbonyloxyalkyl, carbonyloxyaryl, or carbonyloxyalkylaryl group, and

Y is a hydrogen atom or an optionally substituted alkyl, arylalkyl, or aryl group, from a carboxy derivative of structure (2)

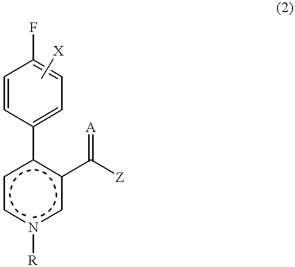

where

A is oxygen or sulphar,

X is one or more of hydrogen, or a readily reducible group,

Z represents either a hydrogen atom or an OY′ group in which Y′ is independently selected from the same groups as Y, and

the broken line circle indicates bonding, appropriate to a tetrahydropyridine, dihydropyridine, pyridine, or piperidine ring

said process comprising

(a) when Y is a hydrogen atom, reducing the compound of structure (2), or

(b) when Y is other than a hydrogen atom

(i) forming an ether from the alcohol product of step (a),

(ii) etherifying the aldehyde compound of structure (2) in which Z is hydrogen, or

(iii) reducing the ester compound of structure (2) in which Z is OY′.

The present invention relates to a new process for preparing pharmaceutically active compounds and intermediates therefor.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxyphenoxymethyl)-piperidine. This compound is used in therapy as the hydrochloride salt to treat inter alia depression, obsessive compulsive disorder (OCD) and panic.

This invention aims to overcome disadvantages in the existing processes for preparation of such compounds and so to provide alternative processes for their manufacture.

This invention has been developed on the basis that compounds of structure (1) below are valuable chemical intermediates useful for the manufacture of important medicinal products, for example paroxetine hydrochloride.

By reference to Example 4 of U.S. Pat. No. 4,007,196, paroxetine may be prepared from a compound of structure (1) below in which R is methyl, and Y is hydrogen, that is 4-(4′-fluorophenyl)-3-hydroxymethyl-1-methylpiperidine, by reaction with 3,4-methylenedioxyphenol followed by demethylation. In the same Example, 4-(4′-fluorophenyl)-3-hydroxymethyl-1-methyl piperidine is prepared by reduction of 4-(4′-fluorophenyl)-3-hydroxymethyl-1-methyl-1,2,3,6-tetrahydropyridine (II), which is in turn prepared from 4-(4′-fluorophenyl)-1-methyl-1,2,3,6-tetrahydropyridine (III), by reaction with formaldehyde.

In EP-A-0152273, compound (II) is prepared by a process in which an α-methyl styrene is reacted with formaldehyde and an amine hydrochloride, via compound (III) as an non-isolated intermediate. Known methods for converting compound (III) into compound (II) are inefficient, and the product (II) has been shown to contain approximately 30% residual compound (III). This is carried forward and causes problems with subsequent stages and with residues in the finally intended product. Furthermore, the use of both hydrochloric acid and formaldehyde can give rise to bis-chloromethylether.

Alternative processes for the preparation of 4-(4′-fluorophenyl)-3-hydroxymethyl-1-methylpiperidine are given in EP-A-0223334, by reduction of compounds of structure (A)

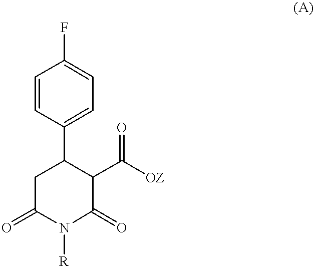

in which Z is alkyl and R is H, alkyl or aralkyl.

The above described processes produce compounds of structure (1) as a mixture of enantiomers. Therefore conversion of compounds of structure (1) to useful pharmaceuticals, such as paroxetine i.e. the (−) trans isomer of 4-(4′-fluorophenyl)-3-(3′,4′-methylenedioxyphenoxymethyl)-piperidine, will normally require a resolution stage, as described in EP-A-0223334.

This invention provides a process for the preparation of a 4-aryl-3-oxymethyl-piperidine of structure (1)

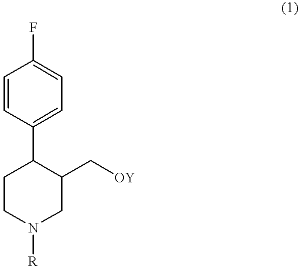

in which R is hydrogen or an alkyl, arylalkyl, allyl, acyl, carbonyloxyalkyl, carbonyloxyaryl, or carbonyloxyalkylaryl group, most suitably lower alkyl or allyl, and Y is a hydrogen atom or an optionally substituted alky, arylalkyl, or aryl group, especially 3,4-methylenedioxyphenyl,

from a carboxy derivative of structure (2)

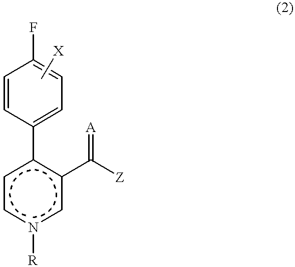

where

A is oxygen or sulphur,

X is one or more of hydrogen, or a readily reducible group such as chlorine, bromine, or iodine,

Z represents either a hydrogen atom or an OY′ group in which Y′ is independently selected from the same groups as Y, and

the broken line circle indicates bonding appropriate to a tetrahydropyridine, dihydropyridine, pyridine or piperdine ring,

said process comprising

(a) when Y is a hydrogen atom, reducing the compound of structure (2), or

(b) when Y is other than a hydrogen atom

(i) forming an ether from the alcohol product of step (a),

(ii) etherifying the aldehyde compound of structure (2) in which Z is hydrogen, before or after reduction of the ring double bonds, or

(iii) reducing the ester compound of structure (2) in which Z is OY′, optionally via an intermediate thioester.

When Y/Y′ is an aryl or alkaryl group, the aryl moiety, for example phenyl, may be optionally substituted by one or more groups such as halogen or alkyl or alkoxy, or by two substituents linked to form a fused ring. For example, an especially suitable substituent Y is 3,4-methylenedioxyphenyl, as found in paroxetine. Alkyl groups, including alkyl groups that are part of other moieties such as alkoxy or acyl, are typically C₁₋₆, especially C₁₋₄ groups, such as methyl and ethyl. Arylalkyl groups are typically benzyl or substituted benzyl. Suitable acyl groups include acetyl and benzoyl and carbonyloxy alkyl groups include methoxycarbonyl, 2-methoxyethyloxycarbonyl, tert.butyloxycarbonyl and phenoxycarbonyl.

Compounds of structure (2) in which A is oxygen are preferred.

Compounds of structure (2) may be either aldehydes or esters or acids of structures (2a) or (2b ).

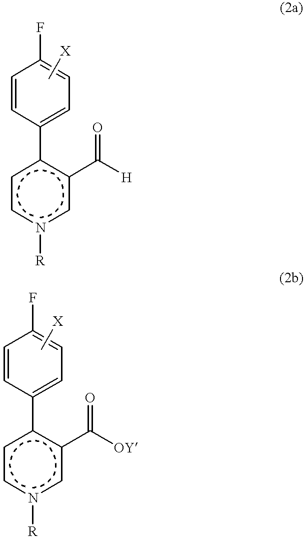

The optional bonds shown by the broken circle mean that the compound of structure (2a) may preferably, inter alia, have the structure (2a1) or (2a2) is tetrahydropyridine and dihydropyridine

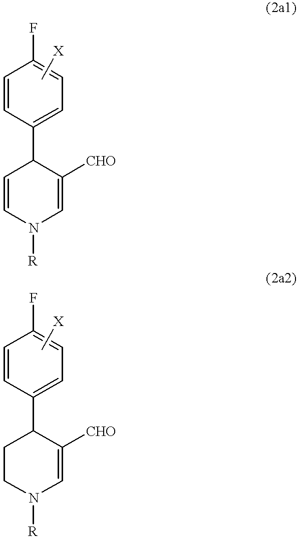

and compounds of structure (2b) may, inter alia, have the structure (2b1) or (2b2)

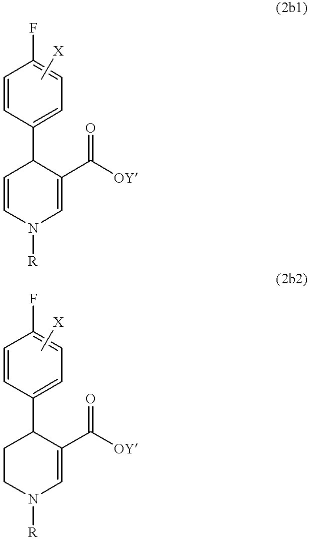

These tetrahydro and dihydro structures are given simply to illustrate the structures encompassed by the broken circle representation in structure (2); all other isomers are also within the scope of the invention.

Compounds of structure (2) are believed to be novel, and form another part of this invention.

It will be appreciated that compounds (2) may be aromatised to pyridine or pyridinium analogues, which may in turn be reduced to (1) or other isomers of (2) by the disclosed methods. The preparation of some pyridine and pyridinium alkyl ester analogues of compound (2) has been described in EP-A-0219934, and some pyridine carbinols and aldehydes and pyridinium carbinols have been described in EP-A-0300617. Reference is directed to EP-A-0219934 and EP-A-0300617 for suitable preparative procedures. Pyridine and pyridinium compounds of structures (6) and (7)

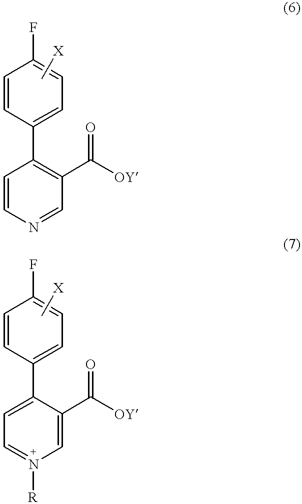

analogous to formula (2) and their conversion to compounds (1) are included in this invention with the exception of the compounds and transformations specifically disclosed above.

In step (a) of the process of this invention, a compound of structure (2) where Z is a hydrogen atom, or a hydroxy, alkoxy, aralkoxy or aryloxy group is reduced to give compounds of structure (1) in which Y is H, that is 3-hydroxymethyl-4-aryl piperidines. This reduction may be accomplished by hydrogenation at atmospheric or above atmospheric pressure using a variety of known catalysts, or using hydride reagents such as lithium aluminium hydride and sodium borohydride, or by a combination of known methods.

For step (b)(i), alkyl, arylalkyl and aryl ethers may be prepared by conventional methods, such as by converting the alcohol into a good leaving group and displacing with a nucleophilic alcohol or alcohol derivative. For example U.S. Pat. No. 4,007,196 describes the conversion of 3-hydroxymethylpiperidine derivatives into methylsulphonate esters and their coupling with 3,4-methylenedioxyphenol in the form of the sodium salt. Alternatively, in step (b)(ii) ethers may be prepared directly by reduction of aldehydes in the presence of alcohols with, for example, trimethylsilane or triethylsilane, optionally catalysed with reagents such as trimethylsilyltrifluoromethanesulphonate or Nafion-H.

In step (b)(iii) of the process of this invention, a compound of structure (2) where Z is other than a hydrogen atom (i.e. Z is OY′) is treated to selectively reduce the carbonyl group and give an ether of structure (1). This may be accomplished by the use of known selective reagents such as diborane and DIBAL, or by Raney nickel desulphurization of a thionoester intermediate, optionally in combination with the methods described for step (a). Thionoesters may be conventionally prepared, for example by treatment of the ester with hydrogen sulphide/chlorotrimethylsilane/lithium di-isopropylamide or Lawesson's reagent. Accordingly compounds of structure (2S) and their transformation to compounds (1) are also part of this invention.

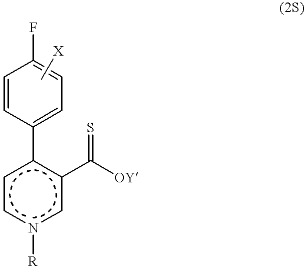

However, it should be noted that direct conversion of dihydropyridines of structure (2a1) to a 3-hydroxymethylpiperidine of structure (1) by conventional reduction methods is not practicable. Although the 5,6 double bond is readily hydrogenated under mild conditions, the 2,3-double bond is unreactive; forcing conditions result in side-reactions such as the elimination of water. It has now been discovered that this difficult hydrogenation may be accomplished by the use of a buffer; an example of a suitable buffer is aqueous sodium acetate. Intermediate compounds of structure (2a2) may be isolated as the products of mild hydrogenation of compounds (2a1).

Compounds of structure (2a) may be prepared by reaction of an appropriately substituted phenyl organometallic derivative with an appropriately substituted pyridine 3-carboxaldehyde. For example, the compound of structure (2a1) where X is a 4-fluoro substituent and R is an acetyl substituent may be made by the reaction of an organometallic derivative of 4-fluorobenzene with pyridine-3-carboxaldehyde, preferably using a Grignard or organocuprate derivative obtained from 4-fluorobromobenzene, followed by acetylation. Advantageously the reaction may be made enantioselective by using the pyridine-3-carboxaldehyde as a derivative with a chiral auxiliary. An example of a suitable chiral auxiliary is 1,2-bis-N-(methylamino)-1(S),2(S)-diphenylethane, which may be recovered for re-use after the coupling reaction by treatment with dilute mineral acid (Alexakis et al., J Org. Chem., 1994, 59, 1877-1888).

When compounds of structure (1) are prepared by reduction of (2) in the absence of a chiral auxiliary, the product will be a mixture of diastereomers, but this need not be a disadvantage if it is to be used in the preparation of paroxetine, since the coupling of N-alkylpiperidines with sesamol may be carried out by a procedure by which both cis and trans isomers react to form the desired trans piperidine ether. N-acyl piperidines are readily reduced to N-alkyl piperidines, for example with lithium aluminium hydride.

It will be appreciated that during reduction of compounds of structure (2) intermediates with structure (3) may be formed and isolated

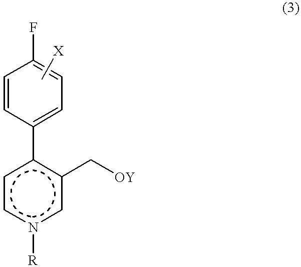

and these compounds with the exception of 4-(4′-fluorophenyl)-3-hydroxymethyl-1-methyl-1,2,3,6-tetrahydropyridine referred to above, when prepared as described above or by other means, are also novel and form part of the invention. Of particular value are compounds of formula (3) where Y is a 3,4-methylenedioxyphenyl group.

Compounds of structure (3) where Y is hydrogen may also be prepared by partial reduction of pyridine carbinols, which may in turn be prepared by hydroxylation of the corresponding methylpyridines by micro-organisms using methods analogous to those described in the Indian Journal of Chemistry (1993) volume 32, page 54.

Certain compounds of structure 2, 3, and 4 may also be prepared by alternative procedures. For example, tetrahydropyridine esters and carbinols and piperidine aldehydes may be prepared by Heck coupling of a substituted iodobenzene to suitable tetrahydropyridine substrates. Hence the Heck coupling of an iodobenzene to a 3-hydroxymethyltetrahydropyridine gives a 4-arylpiperidine-3-carboxaldehyde, to a silyl ether gives a silylated 4-aryl-3-hydroxymethyl-1,4,5,6-tetrahydropyridine, and to a 1,2,5,6-tetrahydropyridine-3-carboxylate gives a 4-aryl-1,4,5,6-tetrahydropyridine-3-carboxylate.

4-aryl-1,2-dihydropyridinecarboxaldehydes of structure (2), for example (2c):

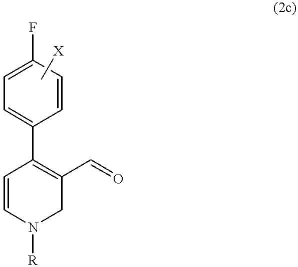

can also be prepared by reduction of isoxazolidines (10)

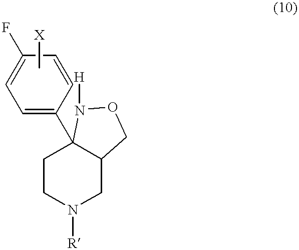

For example, the dihydropyridine (2c) in which X is hydrogen and R′ is ethyl has been prepared by the hydrogenation of the isoxazolidine (10) in which X is hydrogen and R is allyl, in a methanol solution at ambient temperature and pressure using a palladium on carbon catalyst. Compounds of structure (10) in which R′ is hydrogen may be prepared by the reaction of an appropriately substituted organometallic phenyl derivative with an isoxazoline of structure (11).

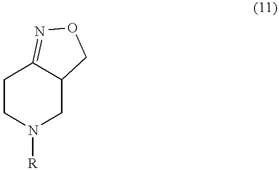

One suitable reagent for this transformation is the lithium derivative obtained from 4-fluorobromobenzene. Other methods for the preparation of compounds of structure (11) include cyclisation of a nitrone obtained from an aryl 3-allylaminoethyl ketone oxime by Grigg methodology.

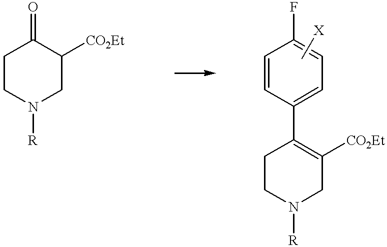

4-aryl-3-alkoxycarbonyl-1,2,5,6-tetrahydropyridines may be prepared by the action of a Grignard reagent derived from, for example, 4-fluorobromobenzene on a 3-alkyloxycarbony 1-4-piperidinone.

Compounds of structure (3) should also be considered to include isomers in which one carbon-carbon double bond is exocyclic, for example (3a):

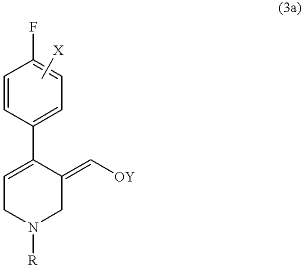

Such compounds may, for example, be prepared by base catalysed condensation of a piperidine-4-one with a formate ester (e.g. ethyl formate) and coupling the resulting enone with a Grignard derivative of a 4-fluorobromobenzene. This may be coupled conventionally with sesamol before reduction to paroxetine. Alternatively, the sesamol coupling may be carried out before the Grignard reaction to give the compound (5), where Y is 3,4-methylenedioxyphenyl,

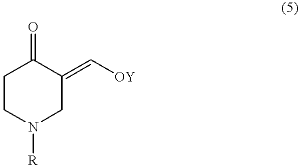

which may then be reacted with the aryl Grignard to give compound (3a), Y is 3,4-methylenedioxyphenyl. Compounds (3a) and (5), where Y has the values defined above, especially Y is hydrogen and Y is 3,4-methylenedioxyphenyl, are also novel and are claimed as part of this invention.

Compounds of structure (1) may be prepared from (3) in the same way as from (2). As with the compounds of formula (2), compounds (3) may also be aromatised to pyridine or pyridinium analogues, which may in turn be reduced to (1) or other isomers of (3) by the disclosed methods; these compounds of structure (8) and (9) and their transformations are also included in this invention.

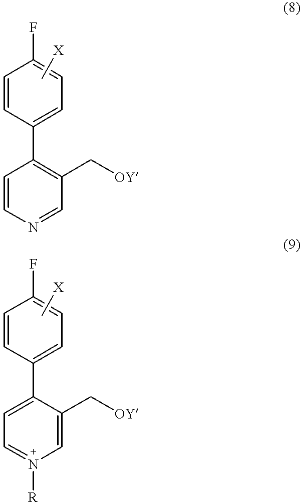

The esters (2b1) may be prepared by methods described in EP-A-0219 934, and may be converted to esters (2b2) by mild hydrogenation.

Compounds (2) may be aromatised to pyridine or pyridinium analogues by methods described in EP-A-0 219 934, and selective reduction of these compounds offers one approach to the preparation of others isomers contained within the general formula (2). An alternative approach to the preparation of pyridine-3-carboxaldehydes for this purpose is described in EP-A-0 300 617.

It will also be appreciated that during reduction of compounds of structure (2) intermediates with structure (4) may be formed and isolated

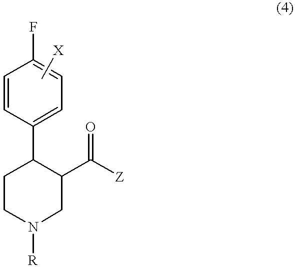

where Z has the same meaning as defined above. Compounds (4) where Z is such as to form an ester with menthol or a lower alkyl group have previously been described. However compounds (4) where Z is such as to form an aldehyde, carboxylic acid, or thionoester or aryl ester, for example with 3,4-methylendioxyphenol, are novel and these compounds when prepared as described above or by other means, are also novel and form part of the invention. Such compounds may be converted into compounds of formula (1) by the same methods as described above, and this conversion is also claimed in this invention.

In a further aspect of the invention, a compound of structure (1) obtained by a process of this invention may be converted to paroxetine using the procedures disclosed in U.S. Pat. No. 3,912,743 or U.S. Pat. No. 4,007,196, typically by condensing a compound of structure (1) in which Y is H with 3,4-methylenedioxyphenol and where necessary removing a group R that is other than hydrogen. When Y is 3,4-methylenedioxyphenyl, then where necessary a group R that is other than hydrogen is removed.

As mentioned above a resolution step may be required during these procedures, unless a chiral auxiliary has been used as previously described.

Paroxetine is preferably obtained as the hydrochloride salt and most preferably as the hemihydrate of that salt, as described in EP-A-0223403. The present invention includes within its scope the compound paroxetine, particularly paroxetine hydrochloride, especially as the hemihydrate, when obtained via any aspect of this invention, and any novel intermediates resulting from the described procedures.

Paroxetine obtained using this invention may be formulated for therapy in the dosage forms described in EP-A-0223403 or WO96/24595, either as solid formulations or as solutions for oral or parenteral use.

Therapeutic uses of paroxetine, especially paroxetine hydrochloride, obtained using this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Accordingly, the present invention also provides:

a pharmaceutical composition for treatment or prophylaxis of the Disorders comprising paroxetine or paroxetine hydrochloride obtained using the process of this invention and a pharmaceutically acceptable carrier,

the use of paroxetine or paroxetine hydrochloride obtained using the process of this invention to manufacture a medicament for the treatment or prophylaxis of the Disorders; and

a method of treating the Disorders which comprises administering an effective or prophylactic amount of paroxetine or paroxetine hydrochloride obtained using the process of this invention to a person suffering from one or more of the disorders.

This invention is illustrated by the following Examples.

### EXAMPLE 1

R-1-acetyl-4-(4′-fluorophenyl)-dihydro-1H-pyridine-3-carboxaldehyde.

4-Bromofluorobenzene (1.40 g) was added dropwise to a stirred suspension of magnesium (0.192 g) in tetrahydrofuran (20 ml) and the mixture heated at reflux for 30 minutes. After cooling to ambient temperature, more tetrahydrofuran (100 ml) was added followed by copper (I) bromide (0.572 g), and the mixture heated at reflux for 30 minutes. The mixture was cooled to ambient temperature and treated with the aminal derived from the reaction of pyridine-3-carboxaldehyde with the chiral auxiliary 1,2-bis-N-(methylamino)-1(R),2(R)-diphenylethane (1.316 g), dissolved in tetrahydrofuran (40 ml). The mixture was further cooled to −78° C., acetyl chloride (0.284 ml, 1 equivalent) added slowly over 20 minutes, stirred at −70° C. for 2 hours, and then allowed to warm up to ambient temperature. Diethyl ether (150 ml) and saturated ammonium chloride which had been made alkaline with ammonia (150 ml) were then added, the phases separated and the aqueous phase extracted with diethyl ether (150 ml). The combined ether phases were washed with saturated sodium chloride (150 ml), dried over anhydrous sodium carbonate, filtered and evaporated. The yield of dihydropyridine aminal was 2.0 g.

R-1-acetyl-4-(4′-fluorophenyl)-dihydro-1H-pyridine-3-carboxaldehyde was isolated from this aminal by treatment with dilute hydrochloric acid and extraction with diethyl ether, and the 1,2-bis-N-(methylamino)-1(R),2(R)-diphenylethane was recovered by basification of the aqueous phase.

### EXAMPLE 2

S-1-acetyl-4-(4′-fluorophenyl)-dihydro-1H-pyridine-3-carboxaldehyde

S-1-acetyl-4-(4′-fluorophenyl)-dihydro-1H-pyridine-3-carboxaldehyde is prepared by an analogous procedure from the pyridine-3-carboxaldehyde aminal formed with 1,2-bis-N-(methylamino)-1(S),2(S)-diphenylethane.

### EXAMPLE 3

4S-1-acetyl-4-(4′-fluorophenyl)-3-hydroxymethylpiperidine.

R-1-acetyl-4-(4′-fluorophenyl)-dihydro-1H-pyridine-3-carboxaldehyde (1.0 g) was dissolved in a mixture of ethanol (100 ml) and 0.1 molar aqueous sodium acetate (100 ml) and hydrogenated at 50 p.s.i. for 18 hours using 10% palladium on carbon catalyst (1.0 g)

The mixture was filtered, evaporated, treated with water (25 ml) and extracted with dichloromethane (100 ml+50 ml). The combined dichloromethane extracts were dried over anhydrous sodium carbonate, filtered and evaporated to give 4S-1-acetyl-4-(4′-fluorophenyl)-3-hydroxymethylpiperidine as a colourless oil.

### EXAMPLE 4

4S-1-methyl-4-(4′-fluorophenyl)-3-hydroxymethylpiperidine.

A solution of 4S-1-acetyl-4-(4′-fluorophenyl)-3-hydroxymethylpiperidine (0.315 g) in tetrahydrofuran (22 ml) was added to a stirred suspension of lithium aluminium hydride (0.143 g) in tetrahydrofuran (6 ml) with cooling in an ice-bath. After the addition was complete the mixture was refluxed for 2 hours and then cooled to room temperature. A mixture of water (1.00 ml) and 15% sodium hydroxide solution (0.143 ml) was added very cautiously, and the resulting suspension stirred at room temperature for 20 minutes. The mixture was filtered and the filtrate evaporated to give the crude product (0.280 g), which was purified by chromatography on silica gel.

### EXAMPLE 5

4-p-Fluorophenyl-3-formyl-1-methylpyridinium bromide

4-p-Fluorophenyl-3-formylpyridine hydrochloride (3.2 g; 8 mmol) was partitioned between saturated aqueous sodium bicarbonate solution (10 ml) and diethylether (10 ml). The aqueous layer was further extracted with ether (3×10 ml) and the combined organic phases were dried over anhydrous magnesium sulphate and evaporated to give the free base (1.42 g; 84%) as a light brown solid. This was dissolved in acetone (10 ml), treated with bromomethane (2 ml; 35 mmol) and left in a stoppered flask at room temperature. After 24 hours a second portion of bromomethane (1 ml; 17.5 mmol) was added and the reaction mixture left to stand at ambient temperature for a further 44 hours. The title compound was isolated by filtration as light brown crystals (1.92 g; 78% yield).

m.p.: 257-258° C.

NMR (d₆-DMSO; Jeol 270 MHz): d10.0. (s) 1H, d9.48 (s) 1H, d9.25 (d) 1H, d8.35 (d) 1H, d7.80 (m) 2H, d7.55 (t) 2H, d4.50 (s) 3H

M/e (FAB) M⁺216

### EXAMPLE 6

(±)4-p-Fluorophenyl-3-hydroxymethyl-1-methylpiperidine

A mixture of 4-p-fluorophenyl-3-formyl-1-methylpyridinium bromide (7.5 mg; 1.7 mmol), platinum dioxide (50 mg) and 10% aqueous ethanol (20 ml) was hydrogenated at atmospheric pressure and at a temperature of 13° C. for 22.5 hours. The mixture was filtered through celite, the solution evaporated under reduced pressure, and the residue treated with saturated aqueous sodium bicarbonate solution (10 ml) and dichloromethane (10 ml). The aqueous phase was further extracted with dichloromethane (2×10 ml), the combined organic phases were dried over anhydrous magnesium sulphate, and evaporated to a brown oil (330 mg). The product was a mixture of cis and trans isomers which were separated by preparative HPLC.

(±)cis-4-p-fluorophenyl-3-hydroxymethyl-1-methylpiperidine:

NMR (CDCl₃; Jeol 270 MHz): d7.2 (m) 2H, d6.9 (t) 2H, d3.55 (m) 2H, d3.1 (dt) 1H, d3.0 (dm) 1H, d2.8 (dt) 1H, d2.45 (m) 2H, d2.0 (s) 3H, d2.05 (td) 2H, d1.7 (m)2H

M/e (CI): M+H 224

(±)trans-4-p-fluorophenyl-3-hvdroxymethyl-1-methylpiperidine:

NMR (CDCl₃; Jeol 270 MHz): d7.2 (m) 2H, d7.0 (t) 2H, d3.4 (dd) 1H, d3.2 (m) 2H, d2.9 (m) 1H, d2.35 (s) 3H, d2.3 (m) 1H, d1.9 (m) 6H

M/e (CI): M+H 224

### EXAMPLE 7

Preparation of 4-(4-fluorophenyl)-1-phenoxycarbonylpiperidine-3-carboxaldehyde.

A mixture of 4-fluoro-iodobenzene (55 mg), 3-hydroxymethyl-1-phenoxycarbonyl-1,2,5,6-tetrahydropyridine (0.29 g), palladium (II) acetate (6 mg), triphenylphosphine (13 mg), and silver phosphate (0.12 g), was stirred in N,N-dimethylformamide (3 ml) at 60° C. for 19 hours under an atmosphere of dry nitrogen. The mixture was cooled to ambient temperature, diluted with diethylether (20 ml) and filtered to remove the solid component. The solid was washed with ether and the combined ether phases extracted with water (2×10 ml). The water phases were then washed with ether and the combined ether phases dried over anhydrous sodium sulphate and evaporated to a pale yellow oil. Yield of title compound 0.32 g.

### EXAMPLE 8

Preparation of 4-(4-fluorophenyl)-3-hydroxymethyl-1-phenoxycarbonylpiperidine.

Sodium borohydride (6 mg) was added to a solution of 4-(4-fluorophenyl)-1-phenoxycarbonylpiperidine-3-carboxaldehyde (56 mg) in dry tetrahydrofuran (2 ml) under an atmosphere of dry nitrogen, and stirred at ambient temperature for 45 minutes. The resulting mixture was treated with water and diethylether, the aqueous phase separated and washed twice with diethylether, and the combined ether phases dried over anhydrous sodium sulphate and evaporated to an oil.

Yield of title compound 55 mg.

### EXAMPLE 9

Preparation of 4-(4-fluorophenyl)-3-methoxycarbonyl-1-phenoxycarbonyl-1,4,5,6-tetrahydropyridine.

A mixture of 4-fluoro-iodobenzene (0.12 g), 3-methoxycarbonyl-1-phenoxycarbonyl -1,2,5,6-tetrahydropyridine (0.67 g), palladium (II) acetate (11 mg), triphenylphosphine (26 mg), and silver carbonate (0.27 g), was stirred in acetonitrile (6 ml) at 80° C. for 15 hours under an atmosphere of dry nitrogen. The mixture was cooled to ambient temperature, diluted with diethylether and filtered to remove the solid component. The solid was washed with ether and the combined ether phases evaporated to a yellow oil.

Yield of title compound 0.74 g.

### EXAMPLE 10

Preparation of 4-(4-fluorophenyl)-3-methoxycarbonyl-1-phenoxycarbonyl-1,4,5,6-tetrahydropyridine.

A mixture of 4-fluoro-iodobenzene (0.11 g), 3-hydroxymethyl-1-phenoxycarbonyl-1,2,5,6-tetrahydropyridine (0.67 g), palladium (II) acetate (11 mg), triphenylphosphine (27 mg), and silver carbonate (0.14 g), was stirred in N,N-dimethylformamide (6 ml) at 60° C. for 40 hours under an atmosphere of dry nitrogen. The mixture was cooled to ambient temperature, diluted with diethylether (10 ml) and filtered to remove the solid component. The solid was washed with ether and the combined ether phases extracted twice with water. The combined ether phases were dried over anhydrous sodium sulphate and evaporated to an oil.

Yield of title compound 0.66 g.

### EXAMPLE 11

Preparation of 3-t-butyldimethylsilyloxymethyl-4-(4-fluorophenyl)-1-phenoxycarbonyl-1,4,5,6-tetrahydropyridine.

A mixture of 4-fluoro-iodobenzene (0.55 mg), 3-t-butyldimethylsilyloxymethyl-1-phenoxycarbonyl-1,2,5,6-tetrahydropyridine (0.43 g), palladium (II) acetate (6 mg), triphenylphosphine (13 mg), and silver phosphate (0.12 g), was stirred in N,N-dimethylformamide (3 ml) at 60° C. for 24 hours under an atmosphere of dry nitrogen. The mixture was cooled to ambient temperature, diluted with diethylether (20 ml) and filtered to remove the solid component. The solid was washed with ether and the combined ether phases extracted twice with water (2×10 ml). The water phases were washed with diethylether (10 ml) and the combined ether phases dried over anhydrous sodium sulphate and evaporated to a pale yellow oil.

Yield of title compound 0.42 g.

### EXAMPLE 12

Preparation of trans-4-(4-fluorophenyl)-piperidine-3-carboxylic acid.

A solution of trans-4-(4-fluorophenyl)-3-ethyloxycarbonylpiperidine (0.51 g, 0.002 mol) in 10 ml ethanol was added to a solution of sodium hydroxide (0.12 g, 0.003 mol) in 95% ethanol. The mixture was brought to reflux and stirred for 1 hour. The solvent was removed in vacuum, the residue neutralized with 10% aqueous hydrochloric acid (1 ml), mixed with toluene and evaporated to dryness. The residue was extracted with hot methanol (25 ml) and the remaining solid material, consisting of inorganic salts, was discarded. The methanol extracts were combined and evaporated under vacuum.

Yield of title compound 0.57 g (76%).

### EXAMPLE 13

Preparation of trans-3-[(3,4-methylenedioxyphenyl)oxycarbonyl]-4-(4-fluorophenyl)-piperidine

A solution of 4-(4-fluorophenyl)-3-ethyloxycarbonylpiperiine (1.22 g, 4.66 mmol, trans/cis ratio 1:1) in ethanol (15 ml) was added to a solution of sodium hydroxide (0.25 g, 6.25 mmol) in 95% ethanol (5 ml). The mixture was brought to reflux and stirred for 1 hour. The solvent was removed under vacuum, the residue of water was removed by distillation with toluene. The solid residue was suspended in chloroform and treated with a solution of thionyl chloride (1.49 g, 12.5 mmol). The mixture was brought to reflux, heated for 1 hour until evolution of sulphur dioxide had ceased, cooled to room temperature, and treated with pyridine (0.99 g, 1.01 ml, 12.5 mmol) followed by a solution of sesamol (0.64 g, 4.66 mmol) in chloroform (5 ml). The mixture was brought to reflux, heated for 6 hours, the solvent removed at reduced pressure and the residue treated with water (5 ml), then extracted with ethyl acetate (20 and 10 ml). The organic extracts were combined, washed with brine (20 ml), dried over anhydrous sodium sulphate, filtered and evaporated to give 3-[(3,4-methylenedioxyphenyl)oxycarbony]-4-(4-fluorophenyl)-piperidine (1.74 g). Corrected yield 60%. Pure 3-[(3,4-methylenedioxyphenyl)oxycarbonyl]-4-(4-fluorophenyl)-piperidine was isolated by column chromatography on silica gel, using a gradient of ethyl acetate/methanol.

### EXAMPLE 14

Preparation of 3-carboxyethyl-4-(4-fluorophenyl)-1,4,5,6-tetrahydropyridine.

Borane-tetrahydrofuran complex (1.0 molar solution in tetrahydrofuran, 10.0 ml, 10.0 mmol) was added by means of a syringe pump over 1 hour to a solution of 3-ethoxycarbonyl-4-(4-fluorophenyl)piperidine-2,6-dione (1.12 g, 4.0 mmol) in 5 ml tetrahydrofuran at 20° C. The reaction mixture was stirred for 3.5 hours and then quenched with 3 ml 50% aqueous potassium carbonate, solution. The phases were separated and the aqueous phase extracted with tetrahydrofuran (25 ml). The organic phases were combined and evaporated to yield 1.25 g of a colorless solid. Separation of the products by column chromatography on 13 g silica gel afforded 0.44 g of the desired product with 93% purity.

Yield 40%.

### EXAMPLE 15

Preparation of 4-(4-fluorophenyl)-3-hydroxymethylpiperidine.

VITRIDE (sodium bis(2-methoxyethoxy)aluminium hydride, 70% solution in toluene, 1.0 ml, 3.5 mmol) was added to a solution of 3-carboxyethyl-4-(4-fluorophenyl)-1,4,5,6-tetrahydropyridine (0.3 g, 1.2 mmol) in tetrahydrofuran (3 ml). The reaction mixture was brought to reflux under a stream of argon gas, which had the effect of removing most of the tetrahydrofuran, and stirred for 2 hours until the starting material had been consumed. The mixture was then cooled to 25° C., quenched with a 50% aqueous potassium carbonate solution (3.5 ml), and filtered. The resulting solid was extracted with ethyl acetate (2×25 ml) and the extracts evaporated to the title compound (0.36 g). This product was purified by column chromatography to yield 4-(4-fluorophenyl)-3-hydroxymethylpiperidine with a cis/trans ratio of 16:84.

### EXAMPLE 16

A solution of acrolein (55 ml) in tetrahydrofuran (50 ml) was added slowly, over 25 minutes, to a solution of diallylamine (90 ml) and DBU (0.88 ml) in tetrahydrofuran (300 ml) at a temperature of approximately −10 C. The mixture was stirred at −15 C. for an hour, at which point a solution of sodium hydroxide (30 g) and hydroxylamine hydrochloride (50 g) in water (200 ml) was slowly added over 30 minutes keeping the temperature below 5 C. n-Hexane (300 ml) was added and the reaction allowed to warm up to ambient temperature with stirring. The mixture was extracted with more n-hexane (300 ml) and the combined organic phases washed twice with water (2×50 ml), dried over anhydrous magnesium sulphate, and evaporated under reduced pressure to a yellow oil (121.2 g). A solution of the yellow oil (1.0 g) in dichloromethane (10 ml) was treated at 20 C. with sodium hypochlorite solution (0.45 g, 8% chlorine equivalent, hence 2.6 ml solution), to give an exothermic reaction. The reaction was monitored by t.l.c. and further portions of sodium hypochlorite were added until the reaction was complete (17.5 ml required in total). The dichloromethane phase was separated, washed with water (5 ml), dried with anhydrous magnesium sulphate, and evaporated. The residue was washed through a silica plug with ethyl acetate, and the solvent evaporated to produce a compound of structure (11), in which R is allyl, as a pale yellow oil (0.47 g).

### EXAMPLE 17

4-fluorobromobenzene (1.32 ml) was dissolved in tetrahydrofuran (18 ml) and slowly treated with tertiary butyl lithium solution (10.85 ml, 1 equivalent) at −78° C. over 10 minutes. The isoxazoline (compound (11) where R is allyl, 1.0 g) in tetrahydrofuran (2 ml) was added over 5 minutes at −78° C. and stirred at this temperature for 2 hours, then allowed to warm to ambient temperature and stirred for a further hour. The mixture was then acidified to pH 2 with hydrochloric acid, diluted with water (20 ml) and extracted with ethyl acetate (20 ml). The aqueous phase was separated, adjusted to pH 9 with aqueous sodium hydroxide solution (2 molar), and extracted twice with ethyl acetate (2×10 ml). The ethyl acetate extracts were combined, dried over anhydrous magnesium sulphate, and evaporated at reduced pressure to produce the isoxazolidine of structure (10) in which R is allyl. Yield 1.28 (82%). Mass spectrum (M+H)⁺, M/Z=263.

What is claimed is:

1. A process for the preparation of a 4-aryl-3-oxymethyl-piperidine of structure (1) 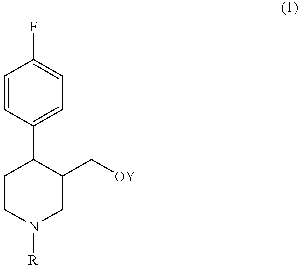
in which R is hydrogen or an alkyl, arylalkyl, allyl, acyl, carbonyloxyalkyl, carbonyloxyaryl, or carbonyloxyalkylaryl group, and
Y is a hydrogen atom or an optionally substituted alkyl, arylalkyl, or aryl group, from a carboxy derivative of structure (2) 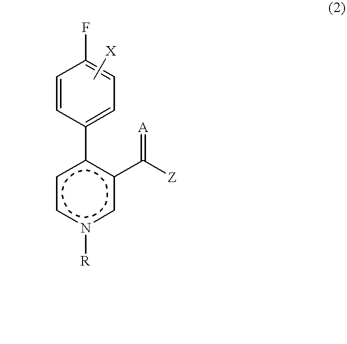
where
A is oxygen or sulphur,
X is one or more of hydrogen, or a readily reducible group,
Z represents either a hydrogen atom or an OY′ group in which Y′ is independently selected from the same groups as Y, and
the broken line circle indicates bonding appropriate to a tetrahydropyridine, dihydropyridine, pyridine, or piperidine ring
said process comprising
(a) when Y is a hydrogen atom, reducing the compound of structure (2), or
(b) when Y is other than a hydrogen atom
(i) forming an ether from the alcohol product of step (a),
(ii) etherifying the aldehyde compound of structure (2) in which Z is hydrogen, or
(iii) reducing the ester compound of structure (2) in which Z is OY′.

2. A process according to claim 1 which is carried out on or via the corresponding tetrahydropyridine or dihydropyridine derivative of the compound of structure (2).

3. A process according to claim 1 which is carried out on or via the corresponding pyridine or piperidine derivative of the compound of structure (2).

4. A process according to claim 1 wherein A in the compound of structure (2) is oxygen.

5. A compound of structure (2).

6. A compound of structure (2S).

7. A compound of structure (3) or (3a).

8. A compound of structure (4).

9. A compound of structure (5).

10. A compound of structure (6).

11. A compound of structure (7).

12. A compound of structure (8).

13. A compound of structure (9).

14. A process according to claim 1 further comprising treating a compound of structure (1) in which Y is hydrogen to condense said compound with 3,4-dioxymethylenephenol, or where Y is other than 3,4-methylenedioxyphenyl converting Y to 3,4-methylenedioxyphenyl, and where necessary removing a group R that is other than hydrogen, so as to obtain a compound of structure (1) in which R is hydrogen and Y is 3,4-methylenedioxyphenyl.

15. A compound of structure (1) where R is hydrogen and Y is 3,4-methylenedioxyphenyl whenever obtained by a process according to claim 1.

16. A compound according to claim 15, in the form of a hydrochloride salt.

17. A pharmaceutical composition for treatment or prophylaxis of the disorders comprising a compound as claimed in claim 15 and a pharmaceutically acceptable carrier.

18. A method of treating the disorders which comprises administering an effective or prophylactic amount of a compound as claimed in claim 15 to a person suffering from one or more of the disorders.
